(19) United States
(12) Patent Application Publication
(10) Pub. No.: US 2019/0142252 A1
(43) Pub. Date: May 16, 2019

(54) OVER-TUBE AND ENDOSCOPIC SYSTEM

(71) Applicant: Olympus Corporation, Tokyo (JP)

(72) Inventor: Tatsuya Higuchi, Tokyo (JP)

(73) Assignee: Olympus Corporation, Tokyo (JP)

(21) Appl. No.: 16/242,790

(22) Filed: Jan. 8, 2019

Related U.S. Application Data
(63) Continuation of application No. PCT/JP2016/079417, filed on Oct. 4, 2016.

(51) Int. Cl. A61B 1/018; A61B 1/00

(52) U.S. Cl. CPC A61B 1/018; A61B 1/00078; A61B 1/00059; A61B 1/00135

(57) ABSTRACT

An endoscopic system includes an over-tube being defined by an elongated tubular member. The elongated tubular member includes an endoscope channel and at least one treatment tool channel each of which is formed longitudinally along a length of the elongated tubular member and is spaced apart from one another within circumference of the elongated tubular member. An endoscope is configured to be inserted into the endoscope channel. Two treatment tools each of which is configured to be inserted into the respective treatment tool channels. A channel position identifier is configured to be disposed in an inner surface of at least a portion of the endoscope channel in a longitudinal direction thereof for indicating the position of the treatment tool channel with respect to the endoscope channel in a circumferential direction. The channel position identifier further includes a transparent window part and an opaque identifying member.

### CROSS-REFERENCE TO RELATED APPLICATION

This application is a continuation application of PCT Application No. PCT/JP2016/079417 filed on Oct. 4, 2016, which is hereby incorporated by reference in its entirety.

### TECHNICAL FIELD

The disclosed technology relates to an over-tube and an endoscopic system.

### DESCRIPTION OF THE RELATED ART

A Japanese Patent Application JP 2013-172780A discloses an over-tube having an identifier that has a characteristic which is added to the shape of an inner surface of a channel in the over-tube. The identifier is visually recognizable with a medical instrument that is inserted in the channel of the over tube in a manner that a user would know position of the medical instrument projected on a distal end of the over-tube.

However, the identifier that has the characteristic is often difficult to be visually recognizable with an observing device inserted in the channel of the over-tube. In particular, it is more difficult to visually recognize the identifier when a transparent liquid such as water or the like is deposited in the channel.

### BRIEF SUMMARY OF EMBODIMENTS

Embodiments of the technology disclosed herein is directed to an over-tube and an endoscopic system constructed so as to assist a user to identify position of a medical instrument inserted in a channel of the over-tube and is projected on the distal end of the over-tube.

According to a first aspect of the technology disclosed, an over-tube comprises an elongated tubular member having at least one treatment tool channel into which a treatment tool is inserted and an endoscope channel into which an endoscope is inserted, and a channel position identifier is disposed in an inner surface of at least a portion of the endoscope channel in a longitudinal direction thereof, for indicating the position in a circumferential direction of the treatment tool channel with respect to the endoscope channel.

In accordance with the first aspect, when the endoscope is inserted into the endoscope channel of the elongated tubular member, the endoscope captures an image of an inner surface of the endoscope channel. The image that is acquired by the endoscope includes an image of the channel position identifier disposed in the inner surface of at least the portion of the endoscope channel in the longitudinal direction thereof. Therefore, the channel position identifier enables the user to confirm the position of the treatment tool channel with respect to the endoscope channel in the circumferential direction. In other words, before the treatment tool that projects through the treatment tool channel from the distal end of the elongated tubular member appears in an endoscope image acquired by the endoscope that projects from the distal end of the tubular member, it is possible to let the user know in which position the treatment tool will appear in the endoscope image.

In the first aspect, the channel position identifier may be disposed in the vicinity of the distal end of the endoscope channel in the longitudinal direction thereof.

With this arrangement, even if the over-tube is twisted and the position of the treatment tool channel with respect to the endoscope channel is different at the proximal and distal ends of the over-tube, it is possible to confirm the position of the treatment tool channel in the circumferential direction in the vicinity of the distal end from which the endoscope is projected, and thereby permitting the user accurately know in which position the treatment tool appears in the endoscope image.

The channel position identifier includes a transparent window part forming as the inner surface of at least the portion of the endoscope channel in the longitudinal direction and an opaque identifying member disposed in a portion in a circumferential direction radially outwardly of the window part.

With this arrangement, when the endoscope is inserted into the endoscope channel and the endoscope captures an image of an inner surface of the endoscope channel, there is acquired an image representing the opaque identifying member disposed in a circumferential direction radially outwardly of the window part through the transparent window part forming as the inner surface of at least the portion of the endoscope channel. As the opaque identifying member is disposed in the portion in the circumferential direction, it is possible to determine the position of the treatment tool channel in the circumferential direction with respect to the endoscope channel from the position of the identifying member in the image.

In the first aspect, the identifying member is disposed between the endoscope channel and the treatment tool channel.

With this arrangement, it determines that the treatment tool channel exists in the position in the circumferential direction where the identifying member exists in the image acquired from the inside of the endoscope channel.

The identifying member is made of a material which is higher in rigidity than the tubular member. The over-tube may further include tensile force transmitting members disposed along the longitudinal direction of the tubular member, for transmitting a tensile force to bend the tubular member. The tensile force transmitting members includes distal ends attached to the identifying member. For example, when a traction force is applied to the proximal end of one of the tensile force transmitting members at one location in a circumferential direction of the tubular member, the tensile force transmitting member transmits a tensile force, pulling the identifying member to which the distal end of the tensile force transmitting member is attached to the proximal side. The portion of the elongated tubular member which is disposed closer to the proximal end of the identifying member is then caused to shrink along the longitudinal direction, thereby bending the tubular member. Since the identifying member is made of a highly rigid material, the identifying member can also be used effectively as a member for bearing tensile forces transmitted by the tensile force transmitting members.

In the first aspect, the identifying member may be disposed to attach the distal ends of the tensile force transmitting members in the vicinity of an outer circumference of the tubular member on both sides of a first straight line which extends through the center of the tubular member and the center of the endoscope channel. With this arrangement, when the tensile force transmitting member attached to the identifying member on either one of the sides of the first straight line is pulled, the tubular member is bent in a direction about the first straight line.

The identifying member may be disposed to attach the distal ends of the tensile force transmitting members on both sides of the first straight line at positions which are offset from a second straight line toward the center of the endoscope channel. The second straight line extends through the center of the tubular member perpendicularly to the first straight line.

With the above arrangement, when two tensile force transmitting members attached to the identifying member on both sides of the first straight line at the positions which are offset from the second straight line toward the center of the endoscope channel are simultaneously pulled, the tubular member is bent in a direction of the endoscope channel about the second straight line.

The over-tube may further include a reinforcing member which increases the torsional rigidity of the tubular member. With this arrangement, the torsional rigidity of the elongated tubular member is increased by the reinforcing member and reducing a twist in the tubular member between its proximal and distal ends. Therefore, even when the channel position identifier is positioned closer to the proximal end of the over-tube, it determines the position of the treatment tool channel in the circumferential direction in the vicinity of the distal end from which the endoscope is projected so as to enable the user accurately knows in which position the treatment tool appears in the endoscope image.

According to a second aspect of the technology disclosed herein, an endoscopic system comprises the over-tube disclosed hereinabove in which an endoscope inserted in the endoscope channel of the over-tube, and a treatment tool inserted in the treatment tool channel of the over-tube.

The present technology offers advantageous effects that enable a user to identify with greater certainty from which position a medical instrument inserted in a channel will project on the distal end of the over-tube.

### BRIEF DESCRIPTION OF THE DRAWINGS

The technology disclosed herein, in accordance with one or more various embodiments, is described in detail with reference to the following figures. The drawings are provided for purposes of illustration only and merely depict typical or example embodiments of the disclosed technology. These drawings are provided to facilitate the reader's understanding of the disclosed technology and shall not be considered limiting of the breadth, scope, or applicability thereof. It should be noted that for clarity and ease of illustration these drawings are not necessarily made to scale.

FIG. 1 is a perspective view depicting a distal end portion of an endoscopic system according to an embodiment described herein.

FIG. 2 is a transverse cross-sectional view of a channel position identifier of an elongated over-tube included in the endoscopic system depicted in FIG. 1.

FIG. 3 is a diagram depicting by way of example an image of the inside of an endoscope channel defined in the over-tube depicted in FIG. 2, which an image is acquired by an endoscope inserted in the endoscope channel.

FIG. 4 is a transverse cross-sectional view of a portion of the over-tube without the channel position identifier.

FIG. 5 is a longitudinal cross-sectional view of a distal end portion of the over-tube depicted in FIG. 2.

FIG. 6 is a transverse cross-sectional view depicting a first modification of the channel position identifier of the over-tube depicted in FIG. 2.

FIG. 7 is a transverse cross-sectional view depicting a second modification of the channel position identifier of the over-tube depicted in FIG. 2.

FIG. 8 is a transverse cross-sectional view depicting a third modification of the channel position identifier of the over-tube depicted in FIG. 2.

FIG. 9 is a transverse cross-sectional view depicting a modification of the over-tube depicted in FIG. 4.

### DETAILED DESCRIPTION OF THE EMBODIMENTS

An endoscopic system 200 having an over-tube 1 according to an embodiment of the technology disclosed herein is described herein after with reference to the drawings.

The endoscopic system 200 includes an endoscope 110, two treatment tools 120, and the over-tube 1.

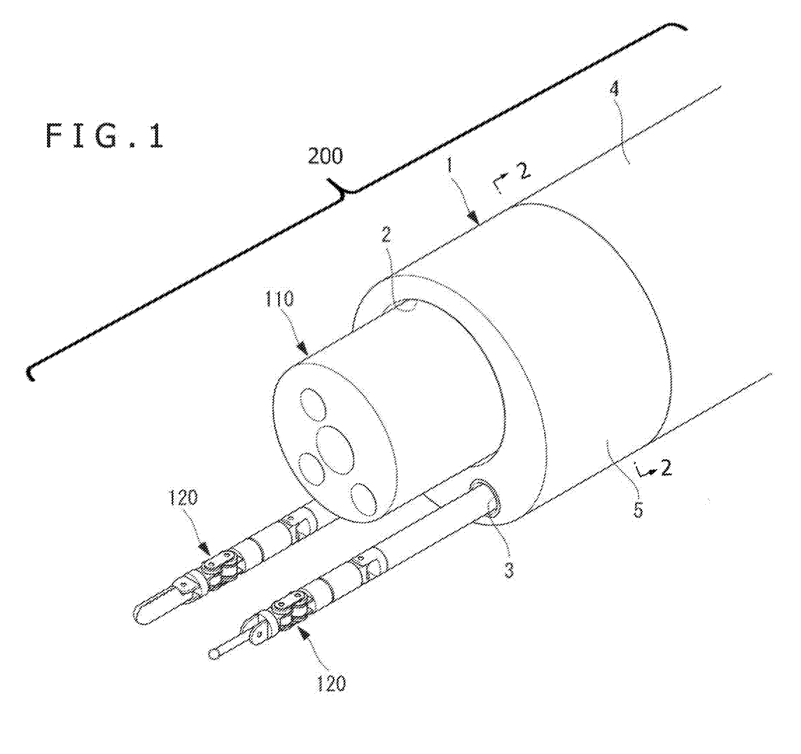

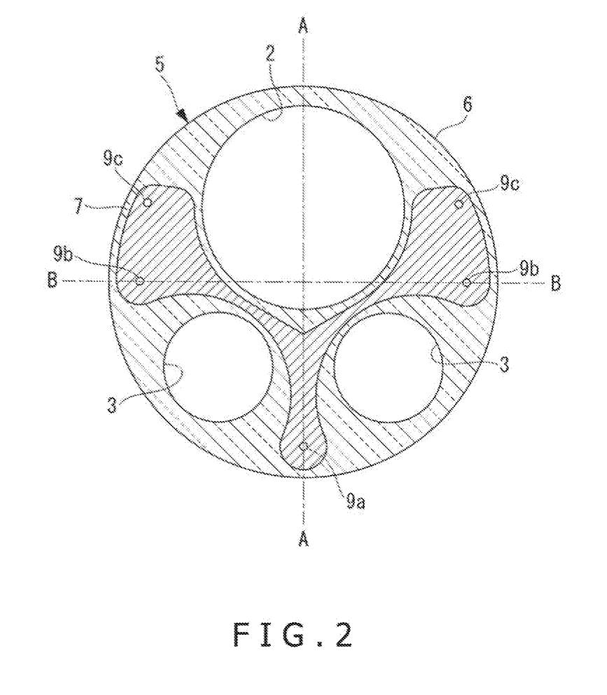

As depicted in FIG. 1, the over-tube 1 includes an elongated tubular member 4 having an endoscope channel 2 and two treatment tool channels 3 all of which formed in a spaced apart relationship with one another and along the longitudinal direction thereof. In one embodiment the over-tube 1 is made of a flexible material, however, one of ordinary skill in the art would appreciate that the over-tube can be made of any material depending on specific construction and operation. An endoscope 110 and respective treatment tools 120 are inserted in the endoscope channel 2 and two treatment tool channels 3, respectively. As depicted in FIG. 2, the endoscope channel 2 and the treatment tool channels 3 in the tubular member 4 are of a circular cross-sectional shape that corresponds to the shape of the respective endoscope 110 and the two treatment tool channels 3. In addition, a channel position identifier 5 is located on the distal end of the over-tube 1.

The channel position identifier 5 is formed by constructing a portion of the over-tube 1 in a longitudinal direction thereof as a window member (window part) 6 made of an optically transparent material, and embedding an identifying member 7 made of an optically opaque material, e.g., flat sheet metal, as depicted in FIG. 2.

The endoscope channel 2 and the treatment tool channels 3 have a circular transverse cross-sectional shape that is steplessly and continuously uniform over the entire length of the tubular member 4.

As depicted in FIG. 2, the identifying member 7 extends between one of the treatment tool channels 3 and the endoscope channel 2, between the other treatment tool channel 3 and the endoscope channel 2, and between the two treatment tool channels 3. The identifying member 7 is substantially Y-shaped as a whole. The portion of the identifying member 7 that extends between the treatment tool channels 3 and the endoscope channel 2 is disposed in covering relation to a substantially semicircular portion of the endoscope channel 2 on the side of the treatment tool channels 3. The identifying member 7 has fixing members 9a, 9b, and 9c for attaching the distal ends of wires (tensile force transmitting members) 8 to be described later, at a position on a first straight line A extending through the center of the elongated tubular member 4 and the center of the endoscope channel 2, position on a second straight line B extending through the center of the elongated tubular member 4 perpendicularly to the first straight line A, and position offset from the second straight line B toward the center of the endoscope channel 2. The fixing members 9a, 9b, and 9c are disposed in the vicinity of the outer circumference of the elongated tubular member 4.

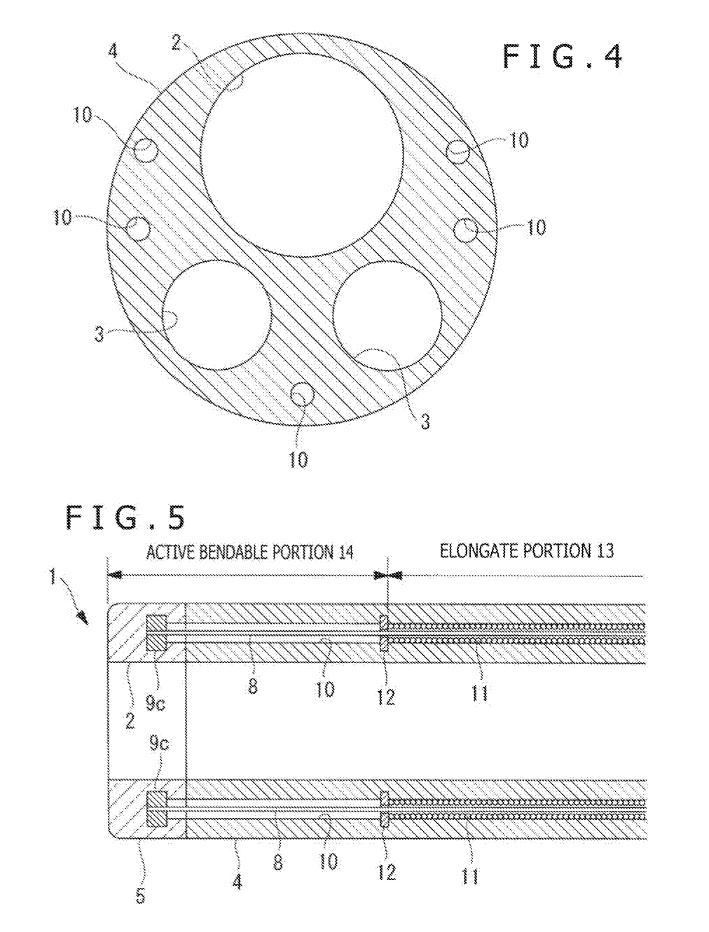

As depicted in FIGS. 4 and 5, the over-tube 1 includes wire channels 10 at positions corresponding to the fixing members 9a, 9b, and 9c in the identifying member 7. The wire channels 10 has an inside diameter sufficiently larger than the outside diameter of the tensile force transmitting wires 8. As depicted in FIG. 5, the tensile force transmitting wires 8 are inserted in the wire channels 10, and have their distal ends fixed to the fixing members 9a, 9b, and 9c in the identifying member 7 that is embedded in the channel position identifier 5.

Coil sheaths 11 are inserted in the wire channels 10 from the distal end of the over-tube 1 to a position that is spaced a predetermined distance from the proximal end of the over-tube 1, and are secured in position by respective sheath retainers 12. The over-tube 1 is thus made up of an elongate portion 13 extending from the sheath retainers 12 toward the proximal end thereof and having its rigidity increased against compressive forces applied to the over-tube 1 along its longitudinal axis. And an active bendable portion 14 extends from the sheath retainers 12 toward the distal end thereof and extensible and contractible when tensile forces are applied to the tensile force transmitting wires 8.

Operation of the over-tube 1 and the endoscopic system 200 thus constructed according to the disclosed embodiment is described hereinafter.

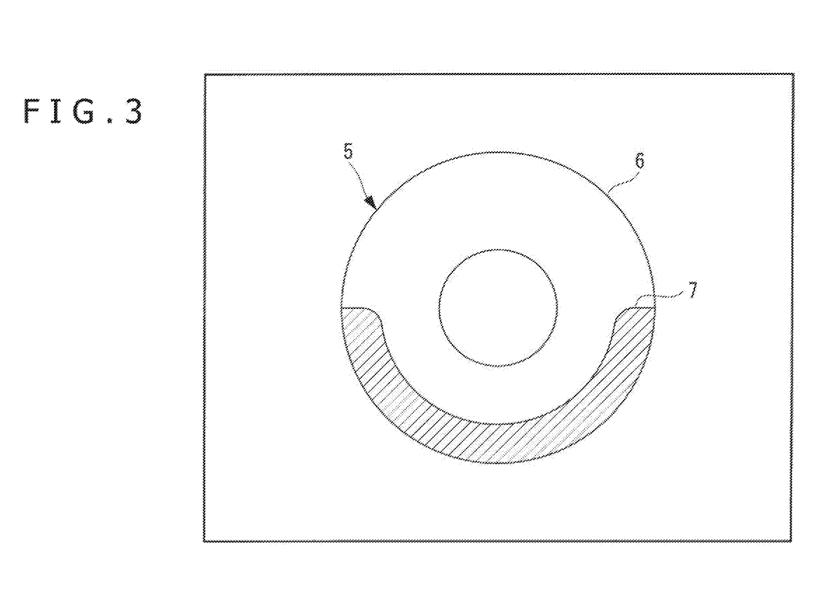

An endoscope 110 is inserted into the endoscope channel 2 in the over-tube 1. When the endoscope 110 is energized, the endoscope 110 acquires an image of an inner surface of the endoscope channel 2. The endoscope 110 is moved forwardly in the endoscope channel 2. When the distal end of the endoscope 110 comes near the channel position identifier 5, the endoscope 110 captures an image of the channel position identifier 5 as depicted in FIG. 3 and stores the image in an image processing unit and display the image on a display monitor. Since the channel position identifier 5 is made up of the optically transparent window member 6 and the optically opaque identifying member 7, the image includes an image of the identifying member 7 (illustrated hatched in FIG. 3) transmitted through the transparent window member 6.

As depicted in FIG. 2, the identifying member 7 is disposed between the endoscope channel 2 and the treatment tool channels 3. Therefore, the user can easily determine that the treatment tool channels 3 exist in the direction of the identifying member 7 in the image depicted in FIG. 3. In other words, with the endoscopic system 200 according to the disclosed embodiment, even though the process of projecting the treatment tools 120 from the distal end of the over-tube 1 and directly capturing an image of the treatment tools 120 with the endoscope 110 as depicted in FIG. 1 is not carried out, the user would know in advance in which positions the treatment tools 120 appear in the image acquired by the endoscope 110.

While the over-tube 1 and the endoscope 110 are being inserted along a meandering path such as a body cavity in a patient, the phase of the endoscope 110 and the over-tube 1 about their longitudinal axes may change due to a twist in the over-tube 1 during the operation of the endoscope. In other words, the position of the treatment tool is changed in the image captured by the endoscope 110 because of the twist. Even in such a situation, the user would recognize the locations where the treatment tools 120 project from the over-tube 1 by confirming the positions where the treatment tools 120 appear in the image before the treatment tools 120 project from the distal end of the over-tube 1.

According to the disclosed embodiment, furthermore, inasmuch as the identifying member 7 is made of a material such as metal or the like which is higher in rigidity than the material of the other portion of the over-tube 1, the identifying member 7 can also be used effectively as a member to which the distal ends of the wires 8 are fixed for bearing tensile forces.

When a traction force is applied to either one of the tensile force transmitting wires 8 fixed to the fixing members 9a, 9b, and 9c at the proximal end of the over-tube 1, the identifying member 7 fixed to the distal end of the over-tube 1 is pulled toward the proximal end thereof under a tensile force transmitted by the wire 8, causing the active bendable portion 14 to bend in the direction toward a side where the wire 8 is located.

If the fixing members 9c is placed in the first straight line A on the side of the endoscope channel 2, the identifying member 7 should be placed all around the endoscope channel 2. In this case, it is difficult to clarify where the treatment tool channels 3 is placed. Therefore, the fixing members 9c for the corresponding wires 8 cannot be placed in the first straight line A on the side of the endoscope channel 2 across the second straight line B in order to keep the identifying member 7 functioning as an identifying member. However, the active bendable portion 14 (shown in FIG. 5) can be bent toward the endoscope channel 2 by simultaneously pulling the two wires 8 that are fixed to the two fixing members 9c that are offset from the second straight line B toward the center of the endoscope channel 2.

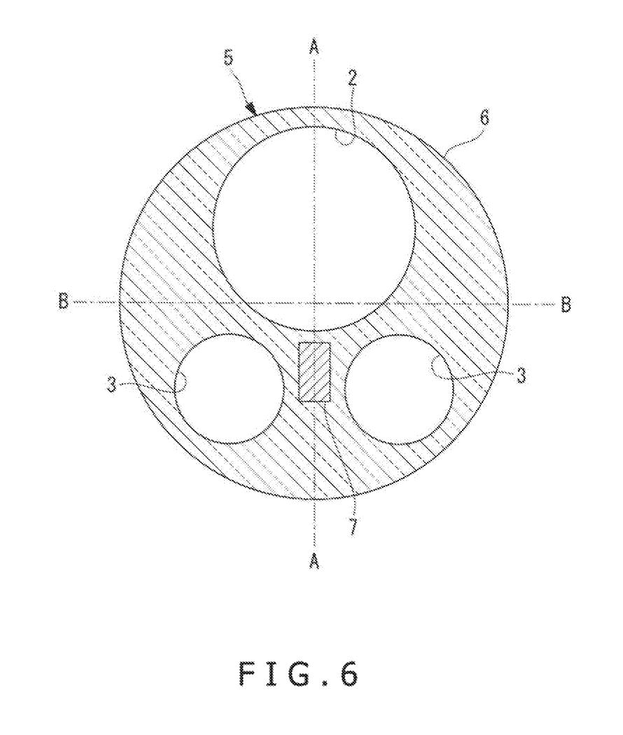

According to the disclosed embodiment, the identifying member 7 is of the Y-shaped as depicted in FIG. 2 so as to use the fixing members 9a, 9b, and 9c not only for identifying the position but also for attaching to the wires 8. However, if the identifying member 7 is to function as an identifying member 7 only, then as depicted in FIG. 6, a simply shaped such as rectangular shaped identifying member 7 may be disposed in a portion near the treatment tool channels 3 across the second straight line B from the endoscope channel 2. In this case, the identifying member 7 may be made of a material of low rigidity.

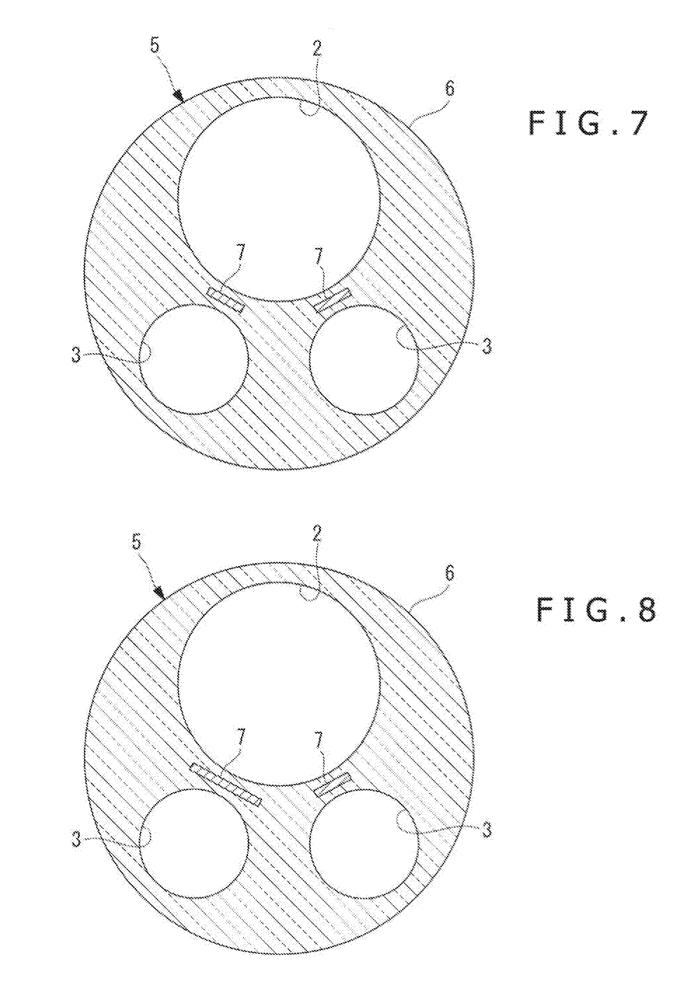

Alternatively, as depicted in FIG. 7, separate identifying members 7 may be disposed in association with the respective treatment tool channels 3. In this case, the identifying members 7 may be of different colors for the respective treatment tool channels 3, or may be of different lengths as depicted in FIG. 8, for distinguishing the treatment tool channels 3 from each other.

Though the arrangements having the two treatment tool channels 3 have been illustrated, the disclosed technology may be applied to an arrangement having three or more treatment tool channels 3 which all of those configurations are within the scope of the disclosed technology. If the treatment tool channels 3 include tubes, then the outer surfaces of the tubes may have different colors, so that the tubes per se that make up the treatment tool channels 3 can be used as identifying members 7.

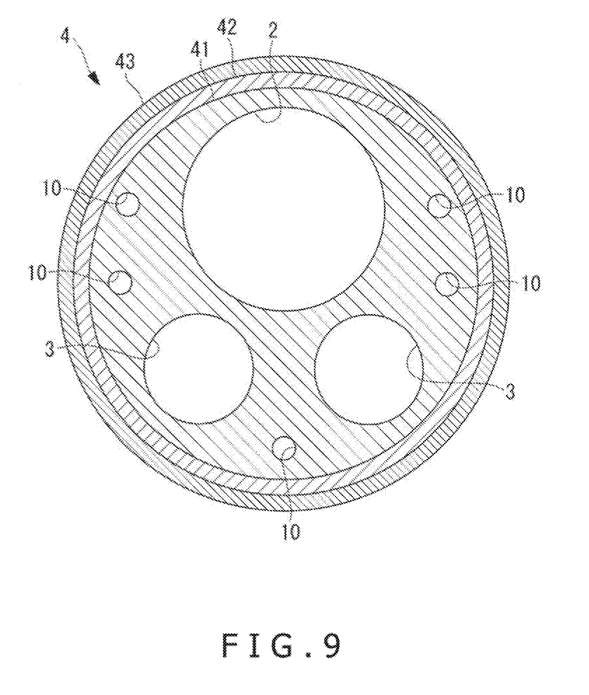

Further alternatively, as depicted in FIG. 9, a tubular member 4 may include a soft multi-lumen tube 41 having treatment tool channels 3 and an endoscope channel 2 therein, a woven tube (reinforcing member) 42 covering the outer circumference of the multi-lumen tube 41, and an outer tube 43 covering the outer circumference of the woven tube 42. The woven tube 42 that covers the soft multi-lumen tube 41 is effective to increase the torsional rigidity of the tubular member 4. Since the tubular member 4 is thus less prone to twist, even if the channel position identifier 5 is positioned closer to the proximal end of the over-tube 1, the user is able to accurately know the positions of the treatment tool channels 3 with respect to the endoscope channel 2.

According to the disclosed technology, the distal end portion of the tubular member 4 is constructed as the window member 6 made of an optically transparent material. However, the window member 6 may be disposed in only a portion of the tubular member 4 in a longitudinal direction thereof.

In sum, one aspect of the disclosed technology is directed to an over-tube comprises an elongated tubular member having an endoscope channel and at least one treatment tool channel each of which being formed in a longitudinal length thereof and are spaced apart from one another. The at least one treatment tool channel receives a treatment tool therein and the endoscope channel receives an endoscope therein. A channel position identifier is configured to be disposed in an inner surface of at least a portion of the endoscope channel in a longitudinal direction thereof for indicating the position of the treatment tool channel with respect to the endoscope channel in a circumferential direction. The channel position identifier is disposed in the vicinity of a distal end of the endoscope channel in the longitudinal direction thereof. The channel position identifier includes a transparent window part and an opaque identifying member. The over-tube further comprises a reinforcing member used to increase the torsional rigidity of the elongated tubular member.

The opaque identifying member is disposed in a part around the endoscope channel. The transparent window part is at least located on an inner side than the opaque identifying member in a circumferential direction of the endoscope channel. The transparent window part is forming as the inner surface of at least the portion of the endoscope channel in the longitudinal direction thereof. The identifying member is disposed between the endoscope channel and the treatment tool channel. The identifying member is made of a material that is higher in rigidity than a material of the elongated tubular member.

The over-tube further comprises a tensile force transmitting member disposed along the longitudinal direction of the elongated tubular member for transmitting a tensile force to bend the elongated tubular member. The tensile force transmitting member includes a distal end attached to the identifying member. The identifying member is disposed to fix the distal end of the tensile force transmitting member in the vicinity of an outer circumference of the tubular member on both sides of a first straight line which extends through the center of the tubular member and the center of the endoscope channel. The identifying member is disposed to fix the distal end of the tensile force transmitting member on both sides of the first straight line at positions which are offset from a second straight line toward the center of the endoscope channel. The second straight line extends through the center of the elongated tubular member perpendicularly to the first straight line.

Another aspect of the disclosed technology is directed an endoscopic system comprises an over-tube defined by an elongated tubular member. The elongated tubular member includes an endoscope channel and at least one treatment tool channel each of which being formed longitudinally along a length of the elongated tubular member and are spaced apart from one another within circumference of the elongated tubular member. An endoscope is configured to be inserted into the endoscope channel. At least one treatment tool is configured to be inserted into the treatment tool channel. The channel position identifier is configured to be disposed in an inner surface of at least a portion of the endoscope channel in a longitudinal direction thereof for indicating the position of the treatment tool channel with respect to the endoscope channel in a circumferential direction. The channel position identifier includes a transparent window part and an opaque identifying member. The transparent window part is forming as the inner surface of at least the portion of the endoscope channel in the longitudinal direction thereof. The opaque identifying member is disposed in a portion in a circumferential direction radially outwardly of the window part. The over-tube further comprises a tensile force transmitting member being disposed along the longitudinal direction of the elongated tubular member for transmitting a tensile force to bend the elongated tubular member. The tensile force transmitting member includes a distal end being attached to the identifying member. The opaque identifying member is Y-shaped so as to enable tensile force transmitting wires to be securely attached to fixing members. The at least one treatment tool channel is defined by two identical treatment tool channels being formed longitudinally along the length of the elongated tubular member to receive respective treatment tools. The over-tube further comprises a reinforcing member being used to increase the torsional rigidity of the elongated tubular member.

While various embodiments of the disclosed technology have been described above, it should be understood that they have been presented by way of example only, and not of limitation. Likewise, the various diagrams may depict an example schematic or other configuration for the disclosed technology, which is done to aid in understanding the features and functionality that can be included in the disclosed technology. The disclosed technology is not restricted to the illustrated example schematic or configurations, but the desired features can be implemented using a variety of alternative illustrations and configurations. Indeed, it will be apparent to one of skill in the art how alternative functional, logical or physical locations and configurations can be implemented to implement the desired features of the technology disclosed herein.

Although the disclosed technology is described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the disclosed technology, whether or not such embodiments are described and whether or not such features are presented as being a part of a described embodiment. Thus, the breadth and scope of the technology disclosed herein should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one”, “one or more” or the like; and adjectives such as “conventional”, “traditional”, “normal”, “standard”, “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more”, “at least”, “but not limited to” or other like phrases in some instances shall not be read to mean that the narrower case is intended or required in instances where such broadening phrases may be absent.

Additionally, the various embodiments set forth herein are described in terms of exemplary schematics, block diagrams, and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular configuration.

What is claimed is:

1. An over-tube comprising:
An elongated tubular member having an endoscope channel and at least one treatment tool channel each of which being formed in a longitudinal length thereof and being spaced apart from one another wherein the at least one treatment tool channel receives a treatment tool therein and the endoscope channel receives an endoscope therein; and
a channel position identifier configured to be disposed in an inner surface of at least a portion of the endoscope channel in a longitudinal direction thereof for indicating the position of the treatment tool channel with respect to the endoscope channel in a circumferential direction.

2. The over-tube of claim 1, wherein the channel position identifier is disposed in the vicinity of a distal end of the endoscope channel in the longitudinal direction thereof.

3. The over-tube of claim 1, wherein the channel position identifier includes a transparent window part and an opaque identifying member wherein the opaque identifying member is disposed in a part around the endoscope channel, the transparent window part is at least located on an inner side than the opaque identifying member in a circumferential direction of the endoscope channel, and the transparent window part is forming as the inner surface of at least the portion of the endoscope channel in the longitudinal direction thereof.

4. The over-tube of claim 3, wherein the identifying member is disposed between the endoscope channel and the treatment tool channel.

5. The over-tube of claim 3, wherein the identifying member is made of a material that is higher in rigidity than a material of the elongated tubular member.

6. The over-tube of claim 1 further comprising:
a tensile force transmitting member being disposed along the longitudinal direction of the elongated tubular member for transmitting a tensile force to bend the elongated tubular member wherein the tensile force transmitting member includes a distal end being attached to the identifying member.

7. The over-tube of claim 6, wherein the identifying member is disposed to fix the distal end of the tensile force transmitting member in the vicinity of an outer circumference of the tubular member on both sides of a first straight line which extends through the center of the tubular member and the center of the endoscope channel.

8. The over-tube of claim 7, wherein the identifying member is disposed to fix the distal end of the tensile force transmitting member on both sides of the first straight line at positions which are offset from a second straight line toward the center of the endoscope channel wherein the second straight line extends through the center of the elongated tubular member perpendicularly to the first straight line.

9. The over-tube of claim 1 further comprising:
a reinforcing member being used to increase the torsional rigidity of the elongated tubular member.

10. An endoscopic system comprising:
An over-tube being defined by an elongated tubular member, wherein
the elongated tubular member having an endoscope channel and at least one treatment tool channel each of which being formed longitudinally along a length of the elongated tubular member and being spaced apart from one another within circumference of the elongated tubular member,
an endoscope being configured to be inserted into the endoscope channel,
at least one treatment tool being configured to be inserted into the treatment tool channel; and
a channel position identifier configured to be disposed in an inner surface of at least a portion of the endoscope channel in a longitudinal direction thereof for indicating the position of the treatment tool channel with respect to the endoscope channel in a circumferential direction.

11. The endoscopic system of claim 10 wherein the channel position identifier includes a transparent window part and an opaque identifying member wherein the transparent window part is forming as the inner surface of at least the portion of the endoscope channel in the longitudinal direction thereof, and the opaque identifying member is disposed in a portion in a circumferential direction radially outwardly of the window part.

12. The endoscopic system of claim 10, wherein the over-tube further comprising a tensile force transmitting member being disposed along the longitudinal direction of the elongated tubular member for transmitting a tensile force to bend the elongated tubular member wherein the tensile force transmitting member includes a distal end being attached to the identifying member.

13. The endoscopic system of claim 11, wherein the opaque identifying member is Y-shaped so as to enable tensile force transmitting wires to be securely attached to fixing members.

14. The endoscope system of claim 11, wherein the opaque identifying member is disposed in a portion close to the treatment tool channels across the second straight line B from the endoscope channel.

15. The endoscope system of claim 11, wherein the at least one treatment tool channel is defined by three or more treatment tool channels and wherein the opaque identifying member is disposed among the three or more treatment tool channels.

16. The endoscopic system of claim 10, wherein the at least one treatment tool channel is defined by two identical treatment tool channels being formed longitudinally along the length of the elongated tubular member to receive respective treatment tools.

17. The endoscopic system of claim 10, wherein the over-tube further comprising a reinforcing member being used to increase the torsional rigidity of the elongated tubular member.
